(19) United States
(12) Patent Application Publication
(10) Pub. No.: US 2026/0090722 A1
(43) Pub. Date: Apr. 2, 2026

(54) SYSTEMS AND METHODS FOR REAL TIME, QUANTITATIVE, HYPERSPECTRAL AND OPTICAL PROPERTY IMAGING FOR IN VIVO INTRAOPERATIVE MULTIPLEX MOLECULAR GUIDED SURGERY

(71) Applicants: Board of Regents, The University of Texas System, Austin, TX (US); William Marsh Rice University, Houston, TX (US); The Brigham and Women’s Hospital, Inc., Boston, MA (US)

(72) Inventors: Pablo Valdes Quevedo, Austin, TX (US); Ashok Veeraraghavan, Houston, TX (US); Alankar Kotwal, Austin, TX (US); Vishwanath Saragadam, Houston, TX (US); Joshua D. Bernstock, Boston, MA (US)

(21) Appl. No.: 19/340,402

(22) Filed: Sep. 25, 2025

Related U.S. Application Data
(60) Provisional application No. 63/700,162, filed on Sep. 27, 2024.

(51) Int. Cl. A61B 5/00

(52) U.S. Cl. CPC A61B 5/0071; A61B 2576/00

(57) ABSTRACT

A method of the present disclosure, among others, involves illuminating a tissue sample with light across both a visible wavelength range and a near-infrared wavelength range, wherein the tissue sample has been administered with at least one fluorescent agent; acquiring a snapshot image of the tissue sample, wherein pixels of the snapshot image capture different 2D views of the tissue sample at a same time along with multiple wavelengths of light incident on a respective sensor pixel; creating a hyperspectral image cube of the tissue sample from the snapshot image; performing spectrally-resolved quantitative fluorescence measurements on the hyperspectral image cube; analyzing the spectrally-resolved quantitative fluorescence measurements to detect a presence of at least one fluorescent agent in the tissue sample; and/or identifying an optical fingerprint of an abnormal region of the tissue sample based on the detected at least one fluorescent agent in the tissue sample.

### CROSS-REFERENCE TO RELATED APPLICATION

This application claims priority to co-pending U.S. provisional application entitled, “Systems and Methods for Real Time, Quantitative, Hyperspectral and Optical Property Imaging for In Vivo Intraoperative Multiplex Molecular Guided Surgery,” having application number 63/700,162, filed Sep. 27, 2024, which is entirely incorporated herein by reference.

### BACKGROUND

In the United States, over 700,000 people live with a primary brain tumor. For these patients, surgery is the initial most effective course of treatment that has been shown to maximize overall survival and quality of life. The neurosurgeon must balance two competing objectives: maximize the extent of resection (EOR), but preserve adjacent normal brain tissue. A maximal EOR can increase overall patient survival but increasing the EOR also increases the risk of damaging normal brain tissue. Surgical disruption of normal brain structures is a major cause of permanent neurologic injury, decreasing overall survival by approximately 40%. In addition, reduced quality of life due to new neurological deficits occur in approximately 30% of patients and any survival advantage provided by maximal EOR is lost if patients incur severe neurologic deficits. Complicating the challenge, primary brain tumors, such as low- and high-grade gliomas, are infiltrative by nature. Tumor cells are found in both the tumor core and the periphery, infiltrating surrounding normal, functional tissues and blurring the boundary between normal and infiltrated brain tissue. This absence of a clear boundary hinders the neurosurgeon in achieving effective and maximally safe surgery. There is a critical need for new technologies capable of helping surgeons maximize EOR while sparing normal brain. This could improve overall patient survival, reduce functional loss for patients, and enhance quality of life.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B show state-of-the-art fluorescence imaging for (A) regions with positive visual fluorescence imaging (vFI) that were detected using a spectrally-resolved spectroscopy probe; and (B) regions with negative vFI that were detected using a spectrally-resolved spectroscopy probe.

FIG. 1C is a diagram depicting light tissue interactions and effects due to absorption and scattering on excitation and fluorescence emissions.

FIG. 1D is a conceptual fluorescence image showing tissue optical phantoms with equal protoporphyrin IX (PpIX) concentrations and varying absorption (μₐ) and scattering (μₛ′) properties which demonstrates how inaccurate vFI assessments are despite compared to quantitative fluorescence imaging (qFI) in phantoms having equal PpIX levels.

FIG. 1E illustrates an example from the inventors from prior literature of an imaging system for dual SSOP reflectance and fluorescence imaging in accordance with the present disclosure.

FIG. 2A-2B show prior literature demonstrations of liquid crystal tunable filter (LCTF)—based hyperspectral fluorescence imaging in rodents and humans.

FIG. 2C shows a table (Table 1) of comparisons between an exemplary hyperspectral quantitative molecular imaging platform of the present disclosure and various existing hyperspectral imaging technologies and their technical specifications as they relate to the clinical application of fluorescence guided surgery.

FIG. 3A is an image of a benchtop hyperspectral imaging (HSI) system for pre-clinical studies for hyperspectral detection in accordance with various embodiments of the present disclosure.

FIG. 3B is an image of a clinical HSI detection add-on module for integration with clinical microscopes in accordance with various embodiments of the present disclosure.

FIGS. 3C-3D show results from using a real-time plenoptic hyperspectral imaging (HSI) system in accordance with various embodiments of the present disclosure.

FIGS. 4A-4D shows previously published results by the inventors for spatial frequency domain (SFDI)/single snapshot optical properties” (SSOP) and SSOP-qFI tissue optical phantoms in accordance with the present disclosure.

FIG. 4E presents an image of a prototype custom exoscope system in accordance with various embodiments of the present disclosure.

FIG. 4F shows output SFDI data for estimation of optical property maps (absorption and scattering) in accordance with the present disclosure.

FIG. 4G shows 3D topographic reconstruction in accordance with various embodiments of the present disclosure that demonstrate real-time quantitative fluorescence-SFDI/SSOP in the VIS-NIR range.

FIGS. 5-6 are block diagrams of exoscopic systems in accordance with various embodiments of the present disclosure.

FIG. 7 is a block diagram of an endoscopic embodiment in accordance with various embodiments of the present disclosure.

### DETAILED DESCRIPTION

The present disclosure provides systems and methods for detecting an ‘optical fingerprint’ of tissues by performing hyperspectral quantitative measurements of light at the surface and deep subsurface, thus enabling exquisite identification of both normal and abnormal (i.e., tumor) tissues.

Existing tools for intraoperative guidance do not offer sufficient information to accurately and practically delineate tumor tissue. Tumor tissue can often be indistinguishable from normal brain parenchyma. Presently, this difficult distinction is made under white light illumination with tactile feedback. Intraoperative neuro-navigation spatially maps the preoperative magnetic resonance imaging (MRI) to help surgeons identify tumor. Even in high-resourced settings, neuro-navigation with pre-operative MRI results in spatial inaccuracies of more than a centimeter due to brain shift occurring during surgery. Intraoperative MRI provides updated information compared to preoperative MRI, but since it is not real-time, it loses spatial accuracy once resection proceeds. Intraoperative ultrasound provides real-time intraoperative feedback with minimal interruption to the surgical workflow but suffers from low sensitivity and specificity. In summary, no surgical adjuncts exist that can reliably help surgeons identify tumor tissues with histologic accuracy during surgery. Therefore, the gold standard remains neuropathologic analysis of fresh frozen tissue as the sole source of intraoperative tissue diagnostic information. This approach is time consuming, requiring the surgeon to remove tissue followed by histological processing and analysis by a neuropathologist that can take up to 30 minutes, and is only available in select well-resourced institutions, creating inequities in outcomes for patients in under or even moderately resourced settings. It is clear that current technologies do not provide sufficient information to help surgeons identify tumor tissue with sufficient specificity and sensitivity during surgery in a timely manner and even the limited tools that exist are not resource-efficient creating inequities in outcomes.

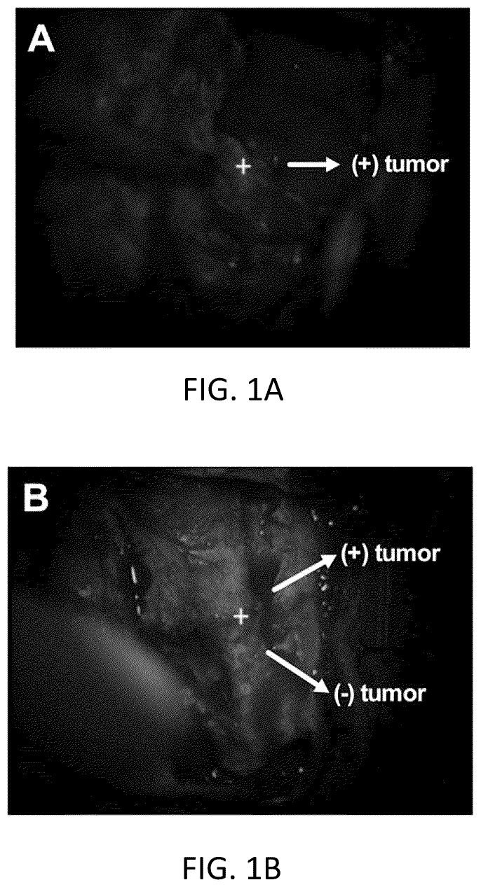

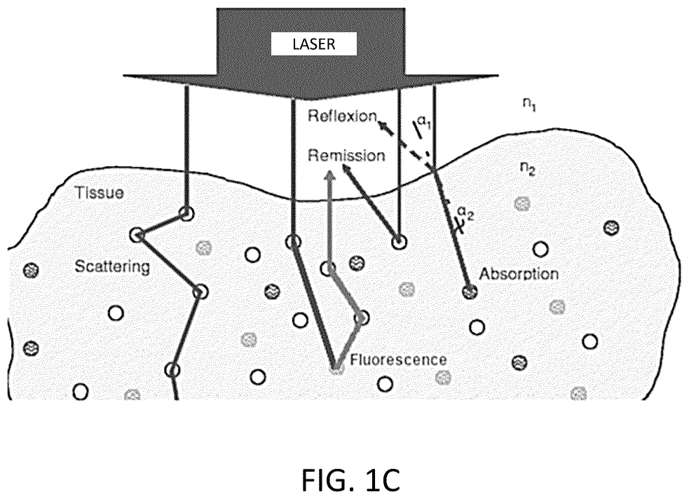

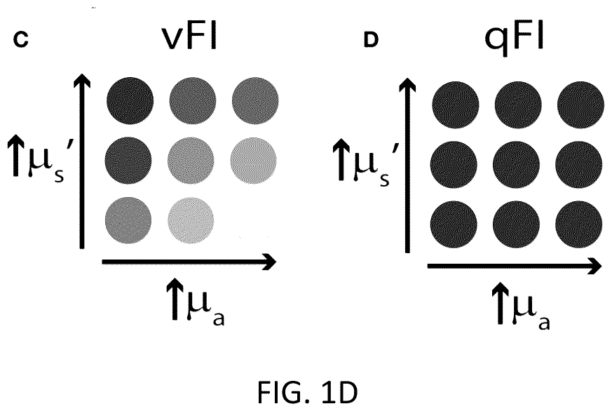

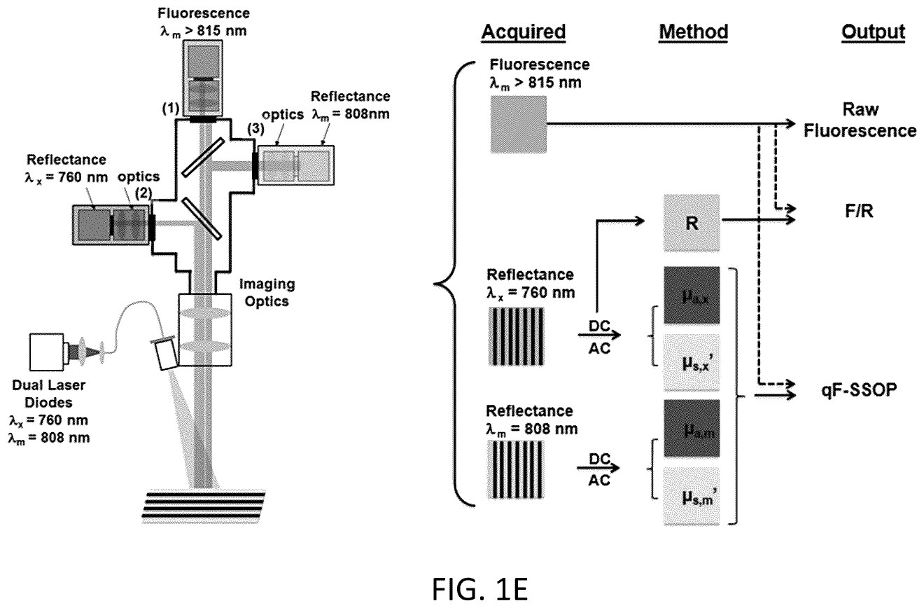

Fluorescence guided surgery (FGS) is a safe and effective surgical adjunct that ‘extends the surgeons vision’ by increasing contrast between tumor and normal tissues. Fundamentally, FGS provides real-time, molecular information at the tissue level using fluorescent biomarkers to distinguish a tumor from normal brain tissue. Accordingly, reference is now made to FIGS. 1A-1B which illustrate state-of-the art fluorescence imaging. Here, the clinical state-of-the-art in FGS for neurosurgical resection of high-grade glioma (HGG) uses FDA-approved 5-aminolevulinic acid (5-ALA)-induced protoporphyrin IX (PpIX) as the fluorescent agent or ‘tumor biomarker’ after being administered to a subject (e.g., via oral or injection administration). The clinical state-of-the-art uses a surgical microscope which has been modified for fluorescence imaging using dedicated optical filters. Such clinical systems provide 405 nm illumination for PpIX excitation and are equipped with a filter that allows light >450 nm to be detected through the surgical oculars and by a color camera. The surgeon then performs a qualitative and subjective assessment of the observed fluorescence, i.e., visual fluorescence imaging (vFI), as shown in FIG. 1A, as high, low, or no fluorescence, as shown in FIG. 1B.

A randomized, multicenter phase III trial evaluated 5-ALA for FGS compared to standard white light guided resection and demonstrated almost doubling of the gross total resection (GTR) rates of contrast enhancing tumor on post-operative MRI (5-ALA group, 65%; white light group, 36%) and progression free survival at 6 months from 21% to 41%. Subsequent studies also demonstrated the utility of PpIX as an intraoperative tumor biomarker that helps improve rates of GTR, which are known to provide improved overall survival and quality of life in patients. FGS provides the surgeon with real-time, molecular information of PpIX in tissue; across the full surgical field of view; without interrupting the surgical workflow; and with high specificity (Sp) and positive predictive values (PPV) >90% for PpIX to distinguish a tumor from normal tissue in HGG.

Despite the positive clinical experience with 5-ALAPpIX FGS, vFI suffers from substantial sensitivity limitations, with higher than ideal false negative rates (i.e., significant tumor is still left unresected) leading to cancer recurrence, and death. That is, in areas where there is positive vFI, surgeons have high confidence there is tumor present (FIG. 1A), but in areas with negative vFI, the surgeon cannot make a confident assessment if the tumor is present or absent (FIG. 1B). For example, the inventors previously confirmed this limitation studying HGG specimens, where they found vFI to have a sensitivity (Sn) and a negative predictive value (NPV) as low as 75% and 26%, respectively. Furthermore, it was also found that for low-grade gliomas (LGGs), vFI provides no surgical value, with Sn of 22% and NPV of 30%. These results highlight the inability of vFI to correctly identify LGGs as well as the infiltrative margins of HGGs, where patients could receive the greatest surgical benefit.

To address these major limitations of vFI, inventor Valdes (Pablo Valdes Quevedo) has shown that the Sn and NPV, and subsequently, the diagnostic performance of 5-ALA-PpIX can be markedly improved via detection of the ‘optical fingerprint’ of tissues by performing spectrally-resolved quantitative fluorescence measurements with a fluorescence spectroscopy (qFS) and a spectrally-resolved (i.e., hyperspectral) fluorescence imaging system. By detecting the ‘optical fingerprint,’ quantitative fluorescence measurements are able to be performed.

Quantitative fluorescence rests on the principle that both the excitation light to illuminate fluorophores and emitted fluorescence light from fluorophores are differentially attenuated as a result of the distorting effects of varying tissue optical properties as light travels through the tissue (i.e. absorption and scattering). Accordingly, as shown in the literature by multiple inventors, FIG. 1C is a diagram depicting light tissue interactions and effects due to absorption and scattering on the excitation and fluorescence emissions. See Gautheron et al., “5-ALA Induced PpIX Fluorescence Spectroscopy in Neurosurgery: A Review,” Front Neurosci., 28 Jan. 2024, Vol. 18-2024. These spatially varying optical properties cause light attenuation (i.e., loss of light) at both excitation and emission wavelengths, which leads to an over- or more importantly, under-estimation of the true fluorophore levels, as depicted by inventor Valdes in the literature as shown in FIG. 1D. See Valdes, et al., “Quantitative Wide-Field Imaging Techniques for Fluorescence Guided Neurosurgery,” Front Surg. (June 6, 2019). Thus, in areas of high attenuation, and despite containing diagnostically significant levels of PpIX, assessments by vFI are inaccurate (i.e., underestimate levels of PpIX), which leaves residual tumor tissue undetected. That is, the residual tumor would have otherwise been detected and removed, had biomarker levels been measured with quantitative fluorescence.

Thus, current FGS methodologies are unable to detect the ‘optical fingerprint’ of tissues, suffering from three major technological flaws (noted below) that limit their utility as an accurate intraoperative surgical adjunct, decrease the EOR and ultimately, impact overall survival and quality of life in patients. Current quantitative and spectrally-resolved approaches in the inventors'experience in over 50 patients, and in the experience of other major centers nationally and internationally have demonstrated the following flaws:
(1) Current FGS Methodologies Cannot Perform Hyperspectral detection: Each Light

signal from tissue used in FGS (e.g., fluorescence light) can be described in terms of its spectral signature (i.e., spectrum) or ‘optical fingerprint’. The light signal detected from normal brain and tumor tissues is a complex combination of fluorescent biomarkers and their unique spectra including the main fluorophore used for FGS (e.g., PpIX) as well as non-specific fluorescence (e.g., photoproducts [Ppp, Product II, Product III], PpIX states (PpIX620, PpIX635), autofluorescence [e.g., NADH, FAD, lipofuscin], exogenous fluorophores [e.g., ICG) and chromophore effects (e.g., oxy-and deoxy-hemoglobin in the brain). However, current FGS technologies cannot perform spectrally-resolved imaging (i.e., do not perform hyperspectral imaging), rather they perform single bandpass or longpass filtering of light which detects only a limited component of the complete ‘optical fingerprint’ of tissues, and thus, are unable to separate the tumor-specific light signal (i.e., PpIX spectrum) from non-specific light signals (i.e., spectra from photoproducts, autofluorescence, chromophores). As a result, no current FGS system is capable of multiplexing (i.e., imaging multiple fluorescent biomarkers simultaneously), despite an ever-growing number of exogenous fluorescent tumor biomarkers in the clinical translation pipeline. If FGS systems performed hyperspectral imaging, they could detect the complete ‘optical fingerprint’ of tissues, unmix/separate each individual fluorescent biomarker, provide greater detection specificity and sensitivity and ultimately, lead to better patient outcomes. The inventors have demonstrated that hyperspectral measurements enables detection of the ‘optical fingerprint’ of tissues; allows for separation of multiple fluorophores simultaneously; and leads to improved and accurate estimates of fluorescent biomarkers (e.g., PpIX).
(2) Current Fgs Methodologies Cannot Measure Tissue Optical properties: Tissues

have intrinsic optical properties (i.e., absorption, scattering) (FIG. 1C) that have a profound effect on how light travels in and out of tissues and thus, on the detected fluorescence. When tissue is illuminated, incoming light is partially lost due to optical properties (i.e., absorption dependent on levels of oxy and deoxy-hemoglobin and scattering dependent on tissue-specific microstructural changes), and only incoming light that is not lost excites fluorescent biomarkers (i.e., PpIX) (FIG. 1C). Once excited, this biomarker emits fluorescent light, which is subsequently partially lost due to optical properties, before finally exiting tissues and being detected (e.g., by a camera or the surgeon's eyes). It is known that every region of a tumor/brain will have different optical properties, especially heterogeneous tumors like HGG. For example, tissue-mimicking optical phantoms with equal PpIX levels but different optical properties can be classified as having “no fluorescence” (FIG. 1D, bottom left “G”) or “high fluorescence” (FIG. 1D, top right “C”). As such, all fluorescence measurements in tissue are fundamentally corrupted by the distorting effects of optical properties. However, current FGS systems cannot measure and therefore, cannot correct for the distorting effects of optical properties on the detected fluorescence. The inventors have proven that performing diffuse reflectance measurements with explicit estimates of optical properties, corrects for their distorting effects on the detected fluorescence; reduces false negatives; and leads to accurate tissue fluorophore estimates (e.g., PpIX) in both HGG and LGG, and ultimately improves FGS accuracy.

(3) Current Fgs Methodologies Cannot Perform Measurements Deep in tissue:

Light signals in the visible (VIS) and near infrared (NIR) range of the spectrum used in FGS only enable surface and subsurface imaging of tissues (˜<250 μm in the VIS and ˜<10 mm in the NIR). The most common agent for FGS in brain tumors is PpIX with excitation (405 nm) and emission (620-710 nm) in the VIS range, which allows for surface imaging. However, ICG is a NIR fluorophore that has shown specific utility for identifying tumors with second window ICG (SWIG) imaging (5 mg/kg ICG dose 24 hours prior to surgery), with excitation (780-808 nm) and emission (800 - 830 nm) in the NIR range, which allows for subsurface imaging >10 mm. In addition, novel tumor targeting fluorescent agents conjugated to ICG are undergoing brain tumor clinical trials. However, current FGS systems cannot simultaneously detect both surface and subsurface fluorescence. The inventors have shown that imaging deeper in tissue enables tumor visualization despite confounding factors such as layers of blood, tissue, or surgical debris, leading to more accurate identification of tumor tissues that would have otherwise been missed.

In accordance with various embodiments of the present disclosure, an exemplary hyperspectral quantitative molecular imaging platform and related methods detect the ‘optical fingerprint’ of tissues by performing hyperspectral quantitative measurements of light at the surface and deep subsurface, thus enabling exquisite identification of both normal and abnormal (i.e., tumor) tissues. In various embodiments, exemplary systems and methods are intraoperative and compatible with FGS, which will have broad translational potential for in vivo surgical guidance leading to improved outcomes in oncologic surgery along with other surgical subspecialties that can benefit from molecular guidance with highly specific optical imaging.

In various embodiments, an exemplary imaging platform or system of the present disclosure is configured to perform snapshot hyperspectral detection of light; real-time optical property measurements; correction of their distorting effects on detected light; and generate a quantitative 3D, profile corrected image of absolute concentrations of tissue biomarkers after the tissue has been administered with fluorescent biomarker agents (e.g., via oral or injection administration to a subject). Pre-clinical studies validate the exemplary system in tissue phantoms and animal brain tumor models to prepare for clinical FGS use.

One innovation of this work, among others, is a molecular image-guidance strategy that enables real-time, highly accurate, intraoperative, 3D-profile corrected, hyperspectral quantitative imaging of multiple fluorescent agents using fluorescence contrast so neurosurgeons can maximize both the resection of brain tumors and the preservation of normal brain tissue. Accordingly, exemplary embodiments utilize advancements in snapshot hyperspectral imaging technologies, spatial frequency domain (SFDI) techniques, FGS infrastructure integration, and quantitative spectrally-resolved fluorescence measurements, as described below:
1. Snapshot Hyperspectral Imaging Technologies for Clinical Surgical Deployment:

The inventors have developed novel and compact (i.e., small form factor) hyperspectral technologies enabling spectrally-resolved measurements in the VIS-NIR range, with high spatial, spectral, and temporal resolution and high sensitivity for intraoperative use as a stand-alone exoscope or microscope add-on system compatible across oncologic surgical subspecialties and other intraoperative surgical needs.
2. Video-rate Tissue Optical Property Imaging Technologies for Clinical Surgical

deployment: The inventors have developed tissue optical property imaging technologies using spatial frequency domain (SFDI) techniques, and specifically, a form of real-time profile corrected SFDI called “single snapshot optical properties” (SSOP) for intraoperative clinical deployment.

In accordance with the present disclosure, FIG. 1E illustrates an imaging system for dual SSOP reflectance and fluorescence imaging, as previously published by inventor Valdes, et al. See Valdes, et al., “qF-SSOP: Real-Time Optical Property Corrected Fluorescence Imaging,” Biomed Opt Express (Jul. 10, 2017). In this example, three temporally and spatially simultaneous images are acquired in real time: one fluorescence (λ>815 nm) and two reflectance images at the excitation (λₓ760 nm) and emission wavelengths (λₘ=808 nm) under SSOP mode. The reflectance images at λₓ and λₘ at one spatial frequency are processed under SSOP conditions to derive the diffuse reflectance and corresponding μₐ and μₛ′ maps. Fluorescence is evaluated using either the raw fluorescence maps only (F_{x,m}) or an attenuation correction is applied unto the raw florescence F_{x,m} to compensate for the distorting effects of tissue optical properties to derive qF-SSOP fluorescence maps (f_{x,m}=F_{x,m}/X).
3. Exemplary hyperspectral quantitative molecular imaging platform is compatible with the FGS infrastructure, paving the way for clinical translation: The inventors have developed the disclosed hyperspectral quantitative molecular imaging platform that integrates real-time VIS-NIR hyperspectral multi-modal fluorescence and SSOP imaging into a hyperspectral quantitative molecular imaging platform compatible with the surgical workflow for VIS-NIR range imaging.
4. Intraoperative, quantitative, hyperspectral, 3D real-time molecular imaging approach for oncologic surgery: Disclosed hyperspectral quantitative molecular imaging platform provides unprecedented quantitative structural and molecular visualization of normal and tumor tissues, which, combined with established adjuncts, will overcome the major limitations facing current FGS, providing a ‘one-size-fits-all fluorophore agnostic’ fluorescence platform for use in other oncologic surgery subspecialties (e.g., breast, lung) and with other fluorescent agents (BLZ-100, FG001, OTL38, LUM015).

From the foregoing, an exemplary hyperspectral quantitative molecular imaging platform of the present disclosure presents a hyperspectral, quantitative, real-time, 3D intraoperative molecular image-guidance surgical adjunct for use during brain tumor surgery. Accordingly, the disclosed system achieves (i.e., high spatial, spectral, and temporal resolution for low light imaging) hyperspectral imaging (HSI) of fluorescence and diffuse reflectance (see step 1 above), real-time measurements of tissue optical properties (see step 2 above), and as a result, simultaneous surface and subsurface detection. Integrating these advances delivers a technology for hyperspectral multi-modal (fluorescence imaging and diffuse reflectance SFDI) quantitative imaging, which enables unprecedented ‘enhanced’ visualization of boundaries between normal brain and tumor tissues (see step 3 above), and with applications in other oncologic surgical subspecialties.

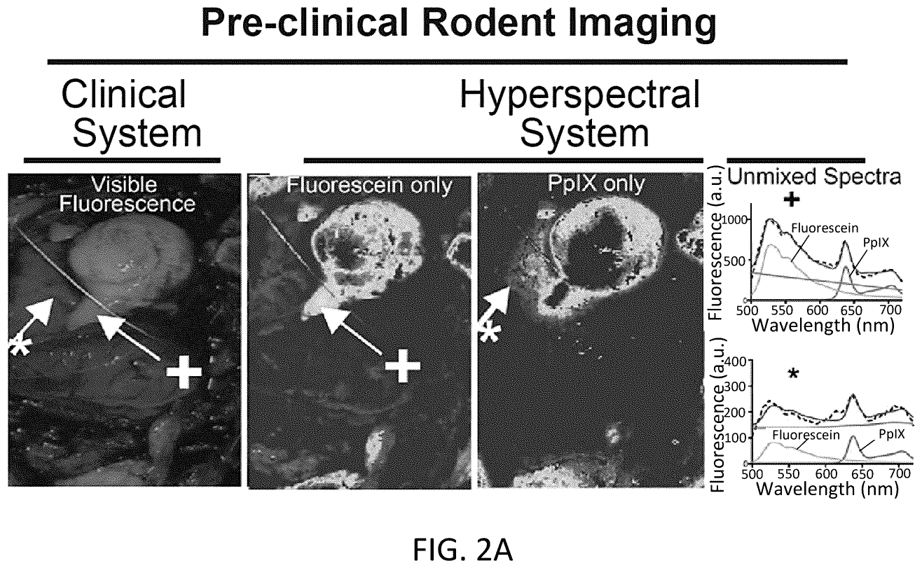

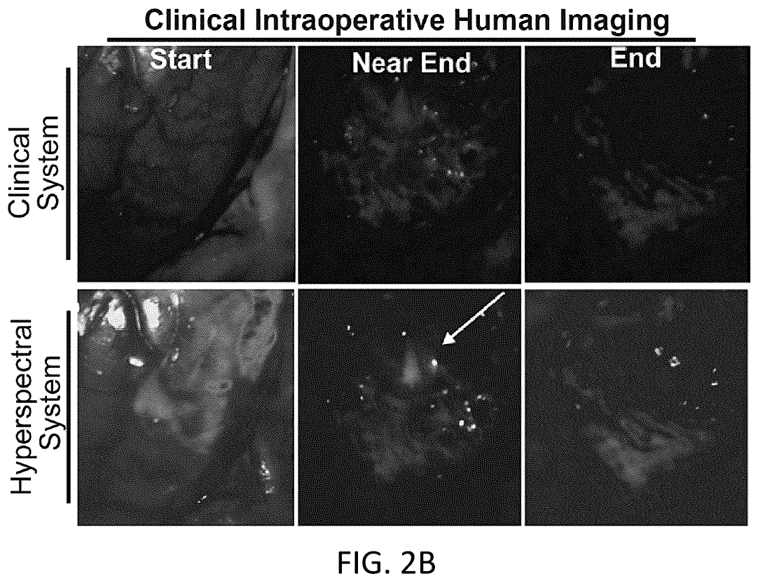

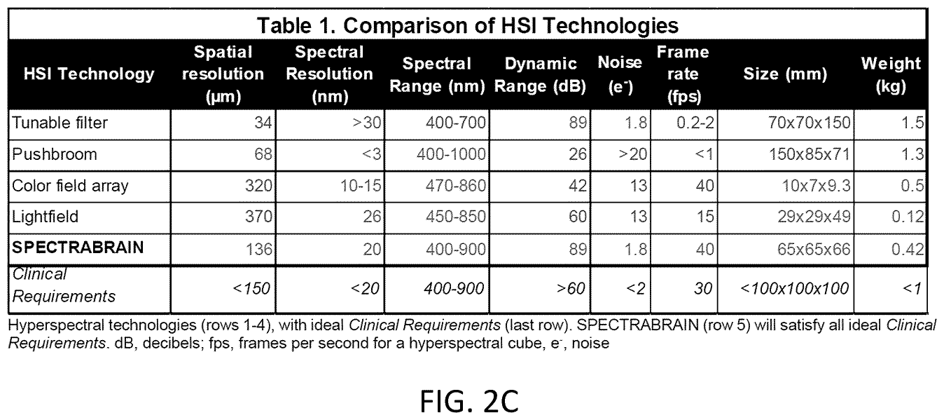

Referring now to FIG. 2A-2B, the figures show prior literature demonstrations of liquid crystal tunable filter (LCTF)—based hyperspectral fluorescence imaging in rodents and humans. For FIG. 2A, hyperspectral fluorescence imaging of 5-ALA-PpIX (red fluorophore) and fluorescein (green fluorophore) fluorescence imaging in a CNS-1 rat glioma model are shown, where only green fluorescence is observed with the clinical system (left image), but HSI enables clear separation of fluorescein (middle image) and PpIX (right image) with spectral unmixing of fluorescein (+, cross) and PpIX (*, asterisks) (far right graphs). Correspondingly, FIG. 2B shows multiple timepoints during Glioblastoma (GBM) surgery at (left column) start of surgery with strong PpIX visible fluorescence with a clinical system (top row) and hyperspectral fluorescence signals (bottom row); near end of resection (middle column) with no visible fluorescence but significant hyperspectral PpIX fluorescence signal confirmed by histopathology and PpIX spectral signature; and at the end of resection (right column) with no visible or hyperspectral fluorescence noted.

In initial pre-clinical and clinical studies, it has been shown that HSI has the potential to significantly improve the distinction between tumor and normal functional brain tissues (FIGS. 2A-2B). For example, HSI has been tested as a surgical adjunct in multiple surgical subspecialties, including neurosurgery. Fundamentally, HSI technologies are designed to create a ‘hyperspectral image cube’, where for each pixel in an image there is a third wavelength (λ) dimension (in addition to x and y coordinates), such that each image pixel contains a spectrum. The inventors have developed a liquid crystal tunable filter (LCTF)-based HSI system that imaged the surgical field in <15 seconds; and have demonstrated in rodent GBM models and in >10 human clinical cases, the feasibility and improved accuracy of single-pixel spectrally-resolved detection across the surgical field of view to separate the individual components from PpIX, fluorescein, and autofluorescence in the VIS range of the spectrum. This LCTF-based HSI approach has been implemented in Germany in >100 cases of LGGs and HGGs, demonstrating HSI had higher accuracy in tissue diagnosis, aided in pharmacodynamic studies, and advanced our understanding of PpIX spectral-states.

Various existing HSI technologies and their technical specifications as they relate to the clinical application of FGS are shown in Table 1 (FIG. 2C), where an exemplary hyperspectral quantitative molecular imaging platform of the present disclosure is referred to as a “SPECTRABRAIN” in row 5 of the table. For the existing HSI technologies, tunable filter systems acquire a single image in time centered at one narrow wavelength band, and then sequentially scan bands over time. Pushbroom systems acquire the full spectrum over a single row in the image, and scan through all the rows to construct the hyperspectral cube. Color field array snapshot systems use prefabricated microfilter arrays attached unto a visible (color) camera sensor to create a hyperspectral cube for fast, but low spatial-resolution acquisition. Lightfield technology-based hyperspectral systems use a microlens array with a small set of spectral filters to create a hyperspectral image at lower spatial resolution.

Clinical studies show that HSI can improve detection of tumor and normal brain regions. Despite these results, current HSI systems are not mainstream due to several major limiting factors/technological challenges, which include large footprint, slow acquisition speeds, low light sensitivity, and low spectral and/or spatial resolution. Thus, for HSI to serve as a surgical adjunct, it requires high spatial resolution (<150 μm), large field of view (7 cm×7 cm), high spectral resolution (<20 nm), wide spectral range (400-900 nm), real-time capture and visualization (>30 fps), small form factor (<1 kg and smaller than 100 mm along each dimension), and high signal to noise (SNR) (>60 dB dynamic range). However, there is no single intraoperative HSI system that has solved these technological challenges (Table 1 (FIG. 2C)). Therefore, there is an acute need for a novel intraoperative HSI technology that addresses these challenges to enable highly accurate and quantitative fluorescence measurements.

In accordance with various embodiments, an exemplary hyperspectral quantitative molecular imaging platform (“SPECTRABRAIN” in row 5 of Table 1 (FIG. 2C)) of the present disclosure integrates an illumination module and a snapshot VIS+NIR hyperspectral sensor with high sensitivity. For illumination, an exemplary system can perform patterned and non-patterned light illumination for SFDI via a custom module having a filtered white light-emitting diode (LED) and 405 nm (VIS) and including but not limited to a 780 or 808 nm (NIR) lasers or LEDs via a custom integrated rigid optics module, liquid light guide and/or fiber optic light delivery setup. In various embodiments, these are coupled to a spatial light modulator for patterned illumination.

In various embodiments, a plenoptic hyper-spectral imager can achieve all the technical specifications required for FGS. In one embodiment, a plenoptic hyperspectral imager has an array of spectral filters in the aperture plane of a light-field camera. The spectral filters modulate the incoming light-field based on the angle of incident light. Then, the various angles of incidence are separately imaged onto the light-field camera allowing effective separation of the various spectral components. Various embodiments of the disclosed system use a plenoptic HSI sensor, which results in lower spatial resolution [370 μm] and high read noise [13e⁻]), as explained below.

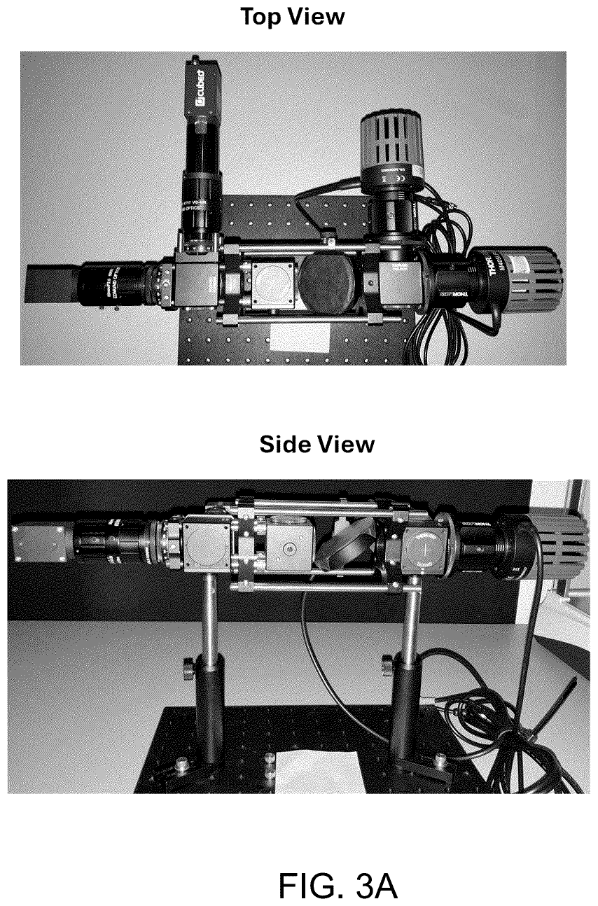

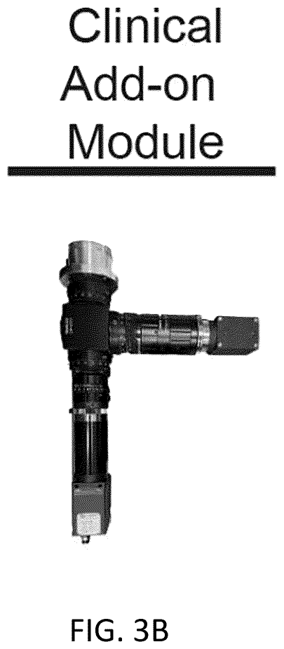

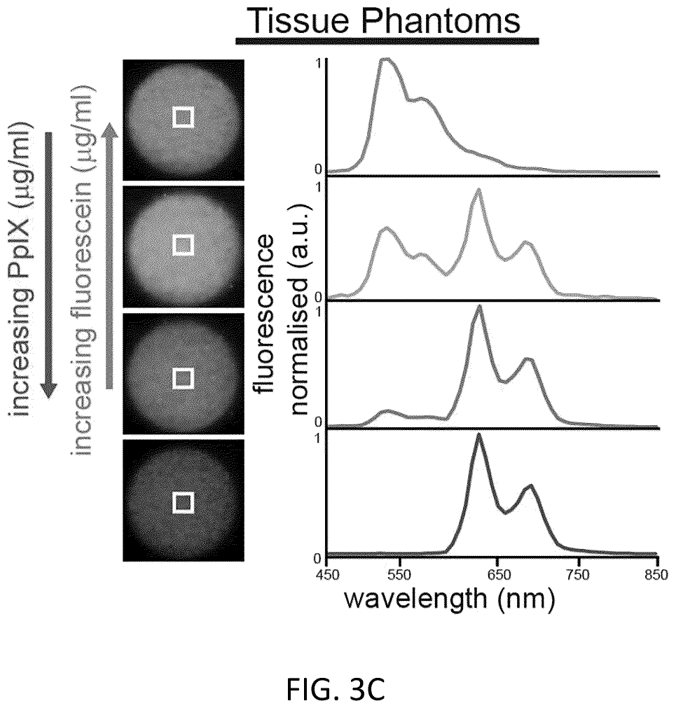

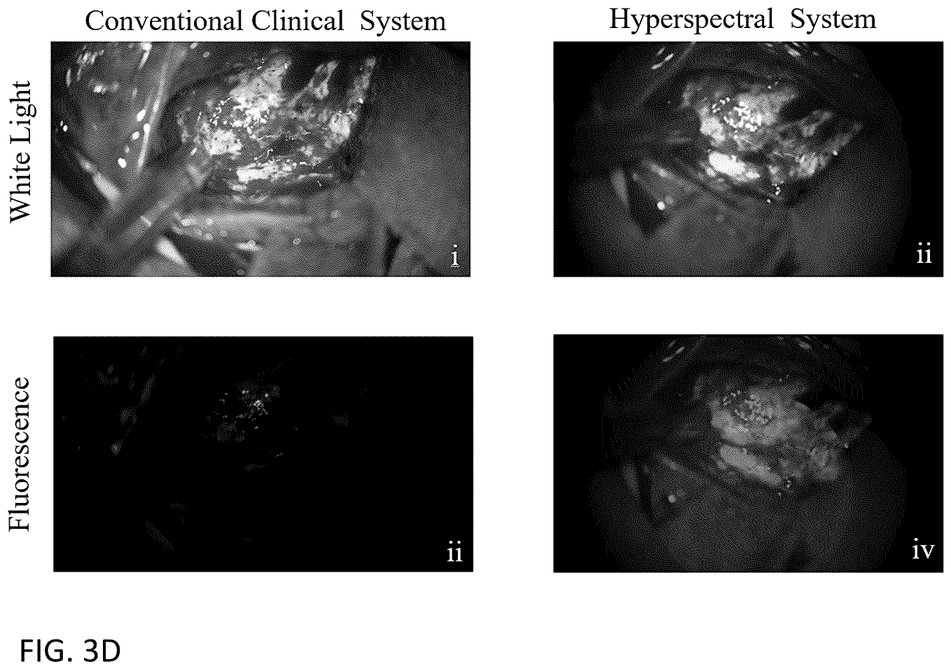

Correspondingly, FIGS. 3A-3D show systems and results from using a real-time plenoptic HSI sensor. In particular, FIG. 3A is an image of a benchtop HSI system for pre-clinical studies for hyperspectral detection in 450-850 nm (VIS-NIR) range and illumination for VIS-NIR clinical fluorophores. FIG. 3B is an image of a clinical HSI detection add-on module for integration with clinical microscopes. FIG. 3C depict tissue simulating phantoms imaged with an HSI plenoptic system with varying concentrations of fluorescein and PpIX (only fluorescein, top row; only PpIX, bottom row; mixed fluorescein/PpIX middle two rows) and corresponding graphs showing spectra from single ROI (white square). FIG. 3D illustrates spectral unmixing following HSI acquired during a human brain tumor surgery following standard of care after ALA administration. Images under white light illumination using a FIG. 3D(i) conventional clinical microscope modified for fluorescence imaging and using the FIG. 3D(ii) HSI system embodied in FIG. 3B. Images under fluorescence illumination conditions (e.g., violet blue light illumination) using a FIG. 3D(iii) conventional clinical microscope and using the FIG. 3D(iv) HSI system embodied in FIG. 3B with green-pseudocolor fluorescence overlay over the white light image following spectral unmixing. The HSI system demonstrates detection of PpIX at levels that were not able to be detected using the conventional system.

Thus, both a pre-clinical benchtop system with integrated custom illumination and detection modules (FIG. 3A), and a compact add-on module that integrates into a surgical microscope (FIG. 3B) have been developed and it was found that the following was able to be achieved: high spectral (<20 nm) and temporal (>15 frames per second [fps]]) resolutions; low light detection for exquisite sensitivity (>20× lower limit of detection than clinical systems); ability to separate/unmix between multiple fluorophores (FIG. 3C) for hyperspectral detection in the VIS-NIR range (450-850 nm); and pre-clinical validation in tissue simulating phantoms (FIG. 3C) and in a human glioma brain tumor surgery following multiplexed hyperspectral fluorescence imaging of PpIX and tissue autofluorescence (FIG. 3D). These promising results demonstrate real-time HSI in the VIS-NIR of diagnostically low fluorophore concentrations in tissue simulating phantoms and in a clinical human brain tumor surgery using the disclosed system.

Accordingly, in various embodiments, an exemplary platform/system performs VIS-NIR imaging using a plenoptic and dedicated spectral-filters and light-field based approach. However, in various embodiments, exemplary systems may also use other spectral separation approaches (e.g., diffraction gratings, programmable spectral filters, etc.).

In one embodiment, VIS-NIR light is detected by a plenoptic HSI Complementary Metal-Oxide-Semiconductor (CMOS) sensor, beamsplitters, filters, and motorized lenses in a custom, small-footprint exoscope system, which allows it to seamlessly integrate into the neurosurgical workflow, given the rapid growth of exoscopes in surgical practice as well as their unique ability for complex data visualization and feedback via digital imaging unlike visualization with traditional microscopes. Further, in various embodiments, an exemplary HSI platform can be utilized as an add-on detection-only module compatible with surgical microscopes for immediate integration and to lower the barrier for adoption (e.g., FIG. 3B). Further, in various embodiments, VIS-NIR light is detected by an exemplary HSI platform by coupling a HSI sensor together with a color (RGB) or monochrome sensor (e.g., CMOS) to enable super-resolved, co-registered imaging of the spatial data with the RGB or monochrome sensor and the spectral data with the plenoptic HSI sensor (e.g., FIG. 3A-3B).

In various embodiments, correction of tissue optical properties may be performed using real time SFDI processing. In an exemplary SFDI technique, special patterns of 2D illumination (e.g., sinusoidal patterns) with different spatial frequencies are captured using an imaging device (e.g., hyperspectral imaging camera). Demodulating algorithms can then be applied to obtain and extract the direct component (DC) (sensitive to changes in reduced scattering and absorption) and alternating component (AC) (sensitive to reduced scattering) of the image.

In such embodiments, profile corrected tissue optical properties of a full surgical field of view can be estimated using a fast acquisition form of SFDI called SSOP imaging. The inventors have previously demonstrated the technical realization of real time measurements of optical properties using SSOP for quantitative fluorescence imaging in tissue optical phantoms, implementing a previously validated (in pre-clinical and clinical studies) fluorescence correction algorithm.

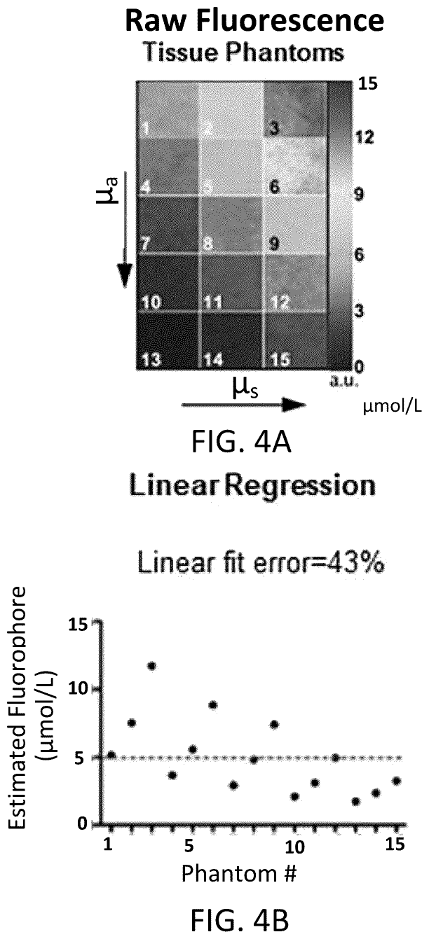

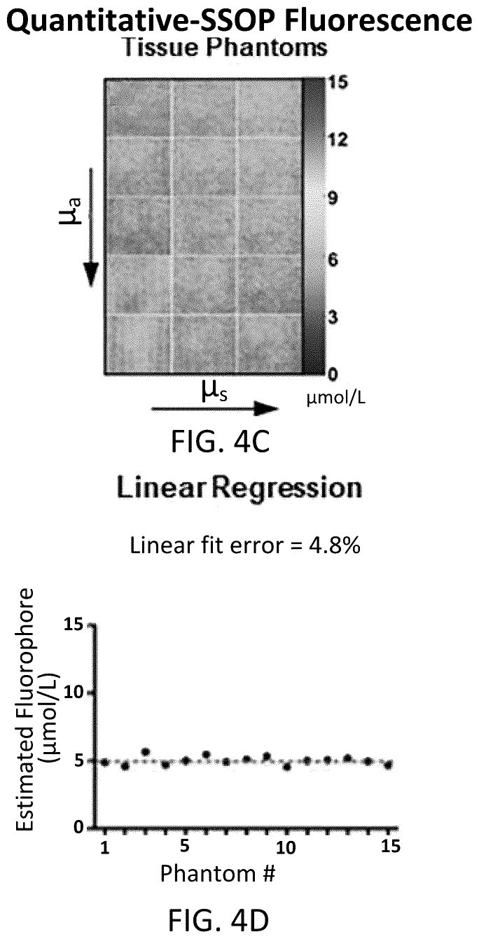

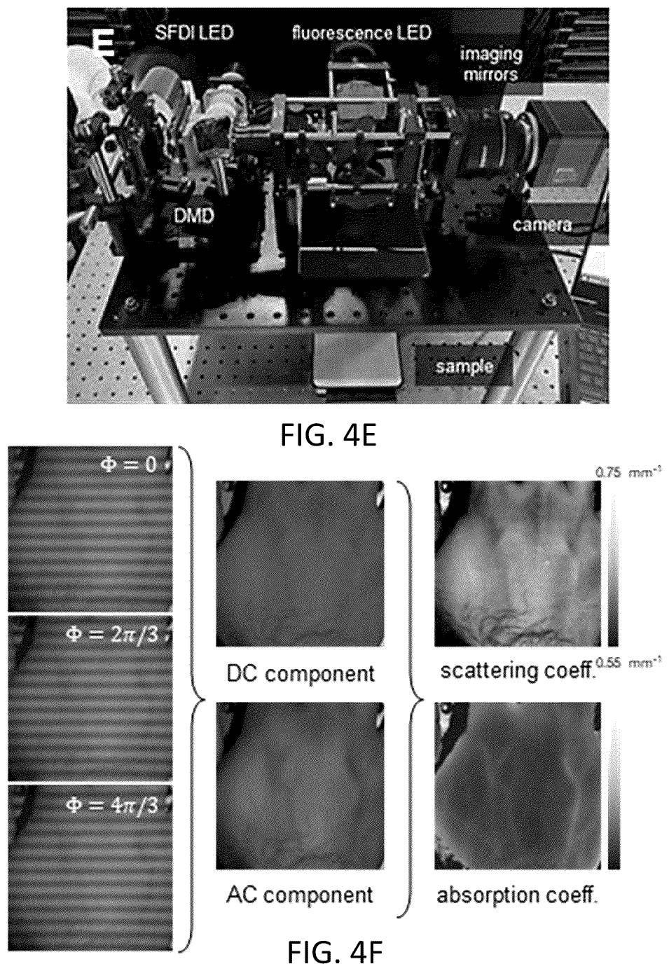

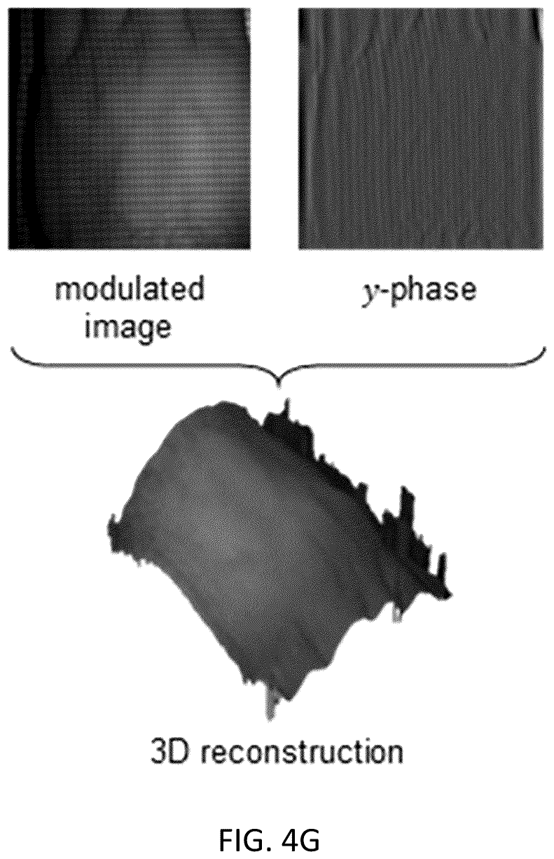

Correspondingly, FIGS. 4A-4D shows previously published results by the inventors of SFDI/SSOP and SSOP-qFI tissue optical phantoms. Here, tissue optical phantoms with carrying absorption (rows) and scattering (columns) properties and the same levels of NIR fluorophore were imaged with a SSOP fluorescence imaging system. FIG. 4A shows raw fluorescence images of phantoms, where FIG. 4C shows SSOP corrected quantitative fluorescence images of phantoms. FIGS. 4B and 4D show corresponding graphs of estimated (y-axis) NIR fluorophore levels versus each phantom with a known NIR fluorophore concentration of 5 μm/L (x-axis) with a significant error in fluorophore estimates using (B) raw fluorescence (error=43%) but (D) accurate NIR fluorophore estimates following correction with quantitative-SSOP fluorescence (error<5%).

FIG. 4E presents an image of a prototype custom SFDI/SSOP exoscope system (individual, opened components in full display) in accordance with various embodiments of the present disclosure. FIG. 4F shows output SFDI (or SSOP if single frequency) data for estimation of optical property maps (absorption and scattering). FIG. 4G shows 3D topographic reconstruction.

Here, it is demonstrated that the detected fluorescence (FIG. 4B, linear fit error =43%) can be corrected for the effects of optical properties to perform highly accurate quantitative fluorescence measurements of fluorophore concentrations (FIG. 4D, linear fit error=4.8%). And, novel results were obtained with a first generation benchtop quantitative fluorescence-SFDI/SSOP exoscope system (FIG. 4E) including a spatial light modulator for patterned illumination (FIG. 4F), scientific CMOS camera, light sources, and custom optics, performing full field of view (7.5 by 7.5 cm), real time (>15 fps) VIS-NIR quantitative fluorescence (400-850 nm) and SSOP imaging to calculate scattering (top), absorption (bottom), and 3D profile maps (FIG. 4G) that demonstrate real-time quantitative fluorescence-SFDI/SSOP in the VIS-NIR with this multi-spectral (i.e., single bandpass/longpass fluorescence imaging) system.

In accordance with various embodiments, 3D SSOP imaging can be performed by projecting two superimposed sinusoidal waves, orthogonal to each other to gather both profile-sensitive pattern orientation (horizontal wave) and optical properties, and profile-insensitive pattern orientation (vertical wave) using the disclosed patterned illumination set up. The profile-sensitive patterns can be used to extract the pixel's height, correcting for height and angle effects, and the profile-insensitive patterns can be used to extract the low frequency, DC (sensitive to changes in reduced scattering and absorption), and high frequency, AC (sensitive to reduced scattering), components of the image.

In various embodiments, a look up table method using Monte Carlo light propagation modeling is performed, where a unique set of optical properties (absorption and reduced scattering) corresponds to a unique set of diffuse reflectance values (at 2 different spatial frequencies), allowing rapid extraction of optical properties and 3D profiles across the full surgical field of view (FIG. 4F). Then, in various embodiments, either a filtered monochrome detection or hyperspectral detection in the VIS-NIR coupled with patterned illumination and exemplary SSOP techniques can be used to calculate optical property maps of absorption and reduced scattering across the 3D field of view. These maps can serve as input into a fluorescence correction algorithm to calculate the quantitative fluorescence in tissue, now devoid of optical property distortions. In various embodiments, a previously validated spectral unmixing method is used to separate individual fluorophore contributions from the corrected hyperspectral fluorescence to derive absolute multiplexed fluorophore concentration estimates.

In various embodiments, an exemplary hyperspectral quantitative molecular imaging platform of the present disclosure integrates (a) hyperspectral detection with multi-wavelength illumination enabling spectrally-resolved imaging of multiple fluorophores in the VIS-NIR range, and (b) SSOP imaging requiring pattern illumination for tissue optical property and 3D profile estimates to achieve real-time, hyperspectral quantitative fluorescence with optical-property and 3D profile corrected imaging.

For validation purposes, the spatial resolution of an exemplary system was assessed using a reflectance USAF 1951 and fluorescence contrast resolution target; optical property measurements were validated using standard commercially available phantoms to enable broad dissemination, standardization, and reproducibility of results; illumination levels were ensured to be below the ANSII limit for light exposure following IEC standards (<185 mW measured 70 mm from light exit with a power meter sensor); fluorescence tissue simulating phantoms of varying, optical properties, and fluorophore (PpIX, ICG) concentrations were constructed; PpIX was used as it is standard of care for gliomas; ICG was used because it is an FDA-approved agent. Novel agents in clinical trials were conjugated to ICG or ICG-similar NIR fluorophores (e.g., BLZ-100 [chlorotoxin-ICG], FG001 [uPAR-binding peptide-ICG], and cetuximab-IRDye800) and are compatible with various embodiments of an exemplary quantitative HSI system. An HSI strategy was tested with simultaneous imaging of 5-ALAPpIX, fluorescence and/or ICG to demonstrate the capabilities for real-time quantitative hyperspectral detection with clinically relevant VIS-NIR fluorophores that will have long-term relevance for FGS in neurosurgery as well as other surgical subspecialities; fluorophore ranges beyond the upper and lower concentration bounds in physiologic and diseased brain states were tested to establish the range of validity for the disclosed system; and system performance was analyzed using linear regression analyses across the range of concentrations to determine linearity of optical property and fluorescence quantification estimates in phantoms.

The published and preliminary results achieved demonstrate how HSI (FIGS. 3A-3D) and SSOP (FIGS. 4A-4G) can be performed for hyperspectral quantitative fluorescence imaging. Here, the present disclosure demonstrates imaging characterization of the disclosed system, which required fluorescence imaging studies. The disclosed system was used to image current clinical agents for FGS in brain tumors (PpIX and fluorescein). The inventors evaluated the HSI system in a human glioma brain tumor surgery (FIG. 3D), with patient's undergoing standard of care fluorescence guided brain tumor surgery. They found that the HSI system is more sensitive to detecting the tumor biomarker, PpIX, compared to conventional clinical fluorescence microscopes and with site biopsy results confirming the presence of tumor.

In accordance with the present disclosure, a transformative new technology is presented for molecular image-guided surgery with a quantitative hyperspectral imaging platform compatible with current and future FGS agents that can substantially improve overall survival and quality of life by enabling surgeons to perform maximally safe brain tumor surgeries. While the present technology demonstrates technological advances in hyperspectral and optical property imaging, the disclosed technology should not be considered to be limited to a current light field approach and may be applied to other applications, such as, but not limited to, camera array microscopes and mesoscopes miniaturized to a point where tens of parallel imaging channels imaged in parallel to achieve the requisite resolution and field of view specifications. It is expected that the disclosed transformative quantitative hyperspectral fluorescence imaging technology will address the critical need of enhancing the surgeon's visualization during surgery to distinguish a tumor from normal tissue by solving the technical challenges of current state-of-the-art fluorescence guidance technologies.

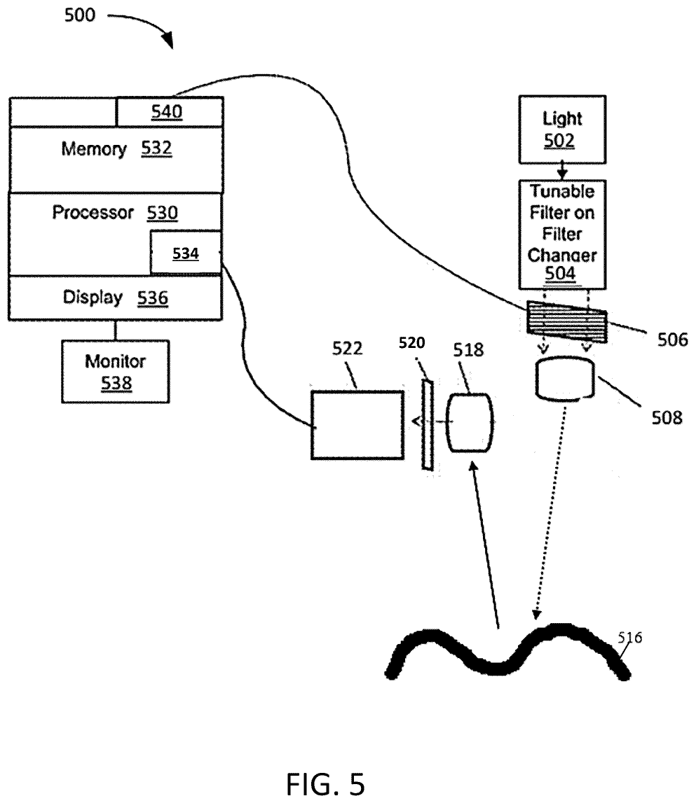

An exoscopic system embodying many of the features and operating modes herein described is illustrated in FIG. 5. In the exoscope 500 of FIG. 5, a supercontinuum laser 502, or similar white light source, or similar set of LEDs, is coupled to pass light through a filter changer 504 that is equipped with a clear filter and a tunable optical bandpass filter, the filter tunable from VIS to NIR nanometer wavelengths with a bandpass. Light from filter-changer 504, which may be filtered light, passes through a spatial modulator 506, such as a modulator based on a digital micromirror device where it is patterned. Light from the spatial modulator passes through a projection lens 508. In an exoscopic embodiment, images of tissue as illuminated by spatially modulated light are processed to determine a surface profile and as such, do not require stereo cameras. At a second, or tissue, the patterned light illuminates tissue 516. Light from tissue 516 is imaged through viewing lens 518 and an optional filter 520 into hyperspectral camera 522 (e.g., hyperspectral plenoptic camera). Signals derived by hyperspectral camera 522 from the light from tissue 516 pass through camera interface 534 and captured images are processed by processor 530 configured with routines in memory 532, the routines having machine readable instructions for performing hyperspectral reflectance imaging, spatially modulated reflectance imaging, fluorescent imaging (including multiple-fluorophore fluorescent imaging), quantitative fluorescent imaging, and depth-resolved fluorescent imaging. Processed images are presented to the surgeon by processor 530 through display adapter 536 on monitor 538. The spatial modulator 506 operates under control of a display adapter 540 under control of processor 530.

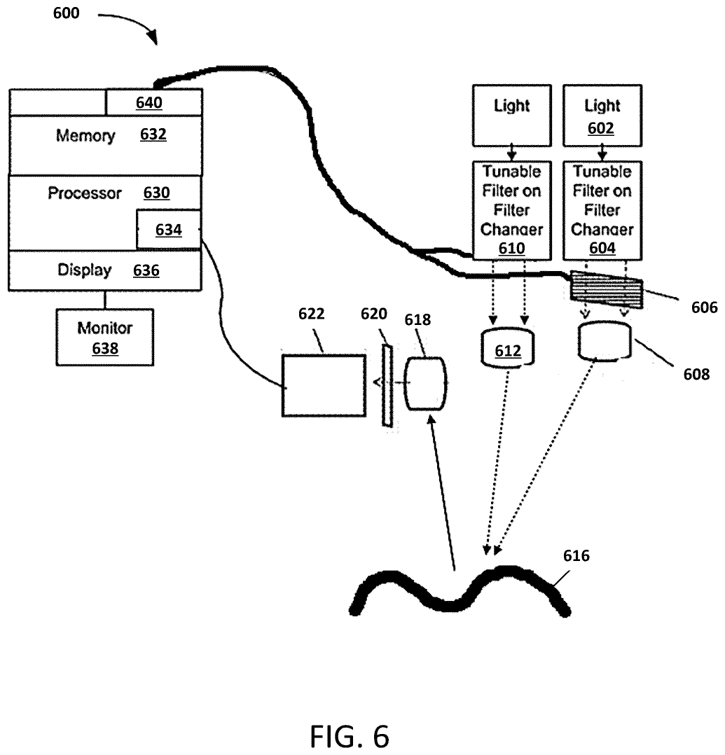

An alternative version of an exoscopic system is illustrated in FIG. 6, in accordance with various embodiments. In the exoscope 600 of FIG. 6, a supercontinuum laser 602, or similar white light source, or similar set of LEDs, is coupled to pass light through a filter changer 604 that is equipped with a clear filter and a tunable optical bandpass filter, the filter tunable from VIS to NIR nanometer wavelengths with a bandpass. Light from filter-changer 604, which may be filtered light, passes through a spatial modulator 606, such as a modulator based on a digital micromirror device where it is patterned. Light from the spatial modulator passes through a projection lens 608. Similarly, light is passed through a filter changer 610 that is equipped with a clear filter and a tunable optical bandpass filter, the filter tunable from VIS to NIR nanometer wavelengths with a bandpass. Light from filter-changer 610, which may be filtered light, passes through a projection lens 612. In an exoscopic embodiment, images of tissue as illuminated by spatially modulated light and/or non-spatially modulated light if the user chooses to use spatial light modulation for hyperspectral imaging, are processed to determine a surface profile and as such, do not require stereo cameras. At a second, or tissue, the patterned light illuminates tissue 616. Light from tissue 616 is imaged through viewing lens 618 and an optional filter 620 into hyperspectral camera 622 (e.g., hyperspectral plenoptic camera). Signals derived by hyperspectral camera 622 from the light from tissue 616 pass through camera interface 634 and captured images are processed by processor 630 configured with routines in memory 632, the routines having machine readable instructions for performing hyperspectral reflectance imaging, spatially modulated reflectance imaging, fluorescent imaging (including multiple-fluorophore fluorescent imaging), quantitative fluorescent imaging, and depth-resolved fluorescent imaging. Processed images are presented to the surgeon by processor 630 through display adapter 636 on monitor 638. The spatial modulator 606 operates under control of the display adapter 640 under control of processor 630.

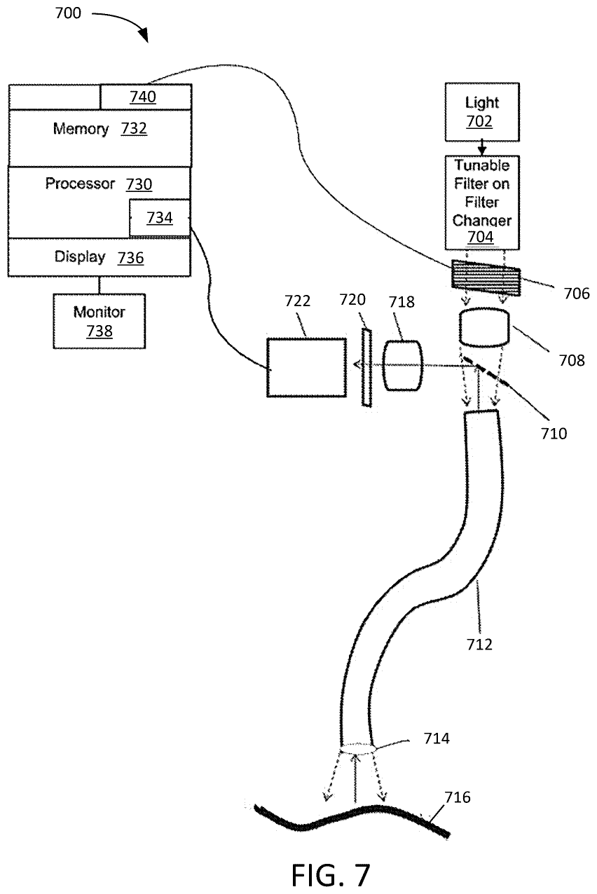

Next, an endoscopic system embodying many of the features and operating modes herein described is illustrated in FIG. 7 as an alternative embodiment. In the endoscope 700 of FIG. 7, a laser 502, or similar white light source, or similar set of LEDs, is coupled to pass light through a filter changer 704 that is equipped with a clear filter and a tunable optical bandpass filter, the filter tunable from VIS to NIR nanometer wavelengths with a bandpass. Light from filter-changer 704, which may be filtered light, passes through a spatial modulator 706, such as a modulator based on a digital micromirror device where it is patterned. Light from the spatial modulator passes through a projection lens 708 and a beam-splitter 710 onto a proximal end of a coherent fiber bundle 712 that runs through the endoscope. In an endoscopic embodiment, images of tissue as illuminated by spatially modulated light are processed to determine a surface profile, since endoscopes typically do not have stereo cameras. At a second, or tissue, end of the coherent fiber bundle the patterned light passes through a tissue-viewing lens 714 onto tissue 716. Light from tissue 716 is imaged through lens 714 and passes onto the tissue end of bundle 712, and is emitted from proximal end of bundle 712 onto beam-splitter 710, where it is diverted through viewing lens 718 and an optional filter 720 into hyperspectral camera 722 (e.g., hyperspectral plenoptic camera). Signals derived by hyperspectral camera 722 from the light from tissue 716 pass through camera interface 734 and captured images are processed by processor 730 configured with routines in memory 732, the routines having machine readable instructions for performing hyperspectral reflectance imaging, spatially modulated reflectance imaging, fluorescent imaging (including multiple-fluorophore fluorescent imaging), quantitative fluorescent imaging, and depth-resolved fluorescent imaging. Processed images are presented to the surgeon by processor 730 through display adapter 736 on monitor 738. The spatial modulator 706 operates under control of a display adapter 740 under control of processor 730.

One or more or more of the components described herein that includes software or program instructions can be embodied in any non-transitory computer-readable medium for use by or in connection with an instruction execution system such as a processor in a computer system or other system. The computer-readable medium can contain, store, or maintain the software or program instructions for use by or in connection with the instruction execution system.

The computer-readable medium can include physical media, such as, magnetic, optical, semiconductor, or other suitable media. Examples of a suitable computer-readable media include, but are not limited to, solid-state drives, magnetic drives, flash memory. Further, any logic or component described herein can be implemented and structured in a variety of ways. One or more components described can be implemented as modules or components of a single application. Further, one or more components described herein can be executed in one computing device or by using multiple computing devices.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y.’”

It should be emphasized that the above-described embodiments of the present disclosed are merely possible examples of implementations, merely set forth for a clear understanding of the principles of the present disclosure. Many variations and modifications may be made to the above-described embodiment(s) without departing substantially from the principles of the present disclosure. All such modifications and variations are intended to be included herein within the scope of this disclosure.

We claim:

1. A method comprising:
illuminating a tissue sample with light across both a visible wavelength range and a near-infrared wavelength range, wherein the tissue sample has been administered with at least one fluorescent agent;
acquiring, via a hyperspectral imaging plenoptic camera sensor, a snapshot image of the tissue sample, wherein pixels of the snapshot image capture different 2D views of the tissue sample at a same time along with multiple wavelengths of light incident on a respective sensor pixel;
creating, via a processor, a hyperspectral image cube of the tissue sample from the snapshot image;
performing, via the processor, spectrally-resolved quantitative fluorescence measurements on the hyperspectral image cube;
analyzing, via the processor, the spectrally-resolved quantitative fluorescence measurements to detect a presence of at least one fluorescent agent in the tissue sample; and
identifying an optical fingerprint of an abnormal region of the tissue sample based on the detected at least one fluorescent agent in the tissue sample.

2. The method of claim 1, wherein multiple fluorescent agents are administered into the tissue sample, and wherein the multiple fluorescent agents are simultaneously detected within the tissue sample.

3. The method of claim 2, wherein the multiple fluorescent agents are simultaneously detected at surface and subsurface levels within the tissue sample.

4. The method of claim 1, wherein the spectrally-resolved quantitative fluorescence measurements correct for distorting effects on the detected at least one fluorescent agent.

5. The method of claim 4, wherein the distorting effects are corrected using spatial frequency domain (SFDI) imaging techniques.

6. The method of claim 4, wherein the distorting effects are corrected using single snapshot optical properties (SSOP) imaging techniques.

7. The method of claim 1, wherein the light is illuminated across the tissue sample at a wavelength range of 450-850 nm.

8. The method of claim 1, wherein an array of spectral filters are positioned in an aperture plane of a light-field camera having the hyperspectral imaging plenoptic camera sensor.

9. A system comprising:
a processor; and
a tangible, non-transitory memory configured to communicate with the processor, the tangible, non-transitory memory having instructions stored thereon that, in response to execution by the processor, cause the processor to perform operations comprising:
causing a tissue sample to be illuminated with light across both a visible wavelength range and a near-infrared wavelength range, wherein the tissue sample has been administered with at least one fluorescent agent;
obtaining, from a hyperspectral imaging plenoptic camera sensor, a snapshot image of the tissue sample, wherein pixels of the snapshot image capture different 2D views of the tissue sample at a same time along with multiple wavelengths of light incident on a respective sensor pixel;
creating a hyperspectral image cube of the tissue sample from the snapshot image;
performing spectrally-resolved quantitative fluorescence measurements on the hyperspectral image cube;
analyzing the spectrally-resolved quantitative fluorescence measurements to detect a presence of at least one fluorescent agent in the tissue sample; and
identifying an optical fingerprint of an abnormal region of the tissue sample based on the detected at least one fluorescent agent in the tissue sample.

10. The system of claim 9, wherein the operations further comprise simultaneously detecting multiple fluorescent agents within the tissue sample.

11. The system of claim 10, wherein the multiple fluorescent agents are simultaneously detected at surface and subsurface levels within the tissue sample.

12. The system of claim 9, wherein the spectrally-resolved quantitative fluorescence measurements correct for distorting effects on the detected at least one fluorescent agent.

13. The system of claim 12, wherein the distorting effects are corrected using spatial frequency domain (SFDI) imaging techniques.

14. The system of claim 12, wherein the distorting effects are corrected using single snapshot optical properties (SSOP) imaging techniques.

15. The system of claim 9, wherein the light illuminated across the tissue sample is within a wavelength range of 450-850 nm.

16. The system of claim 9, further comprising an array of spectral filters positioned in an aperture plane of a light-field camera having the hyperspectral imaging plenoptic camera sensor.

17. The system of claim 9, wherein the processor is integrated within a stand-alone exoscope.

18. The system of claim 9, wherein the processor is integrated within an add-on module for a clinical microscope.

19. A non-transitory computer-readable medium having instructions stored therein, wherein the instructions, when executed by a processor, cause the processor to:
cause a tissue sample to be illuminated with light across both a visible wavelength range and a near-infrared wavelength range, wherein the tissue sample has been administered with at least one fluorescent agent;
obtain, from a hyperspectral imaging plenoptic camera sensor, a snapshot image of the tissue sample, wherein pixels of the snapshot image capture different 2D views of the tissue sample at a same time along with multiple wavelengths of light incident on a respective sensor pixel;
create a hyperspectral image cube of the tissue sample from the snapshot image;
perform spectrally-resolved quantitative fluorescence measurements on the hyperspectral image cube;
analyze the spectrally-resolved quantitative fluorescence measurements to detect a presence of at least one fluorescent agent in the tissue sample; and
identify an optical fingerprint of an abnormal region of the tissue sample based on the detected at least one fluorescent agent in the tissue sample.

20. The non-transitory computer-readable medium of claim 19, wherein the instructions further cause the processor to simultaneously detect multiple fluorescent agents within the tissue sample.
